(19) United States
(12) Patent Application Publication
(10) Pub. No.: US 2020/0337912 A1
(43) Pub. Date: Oct. 29, 2020

(54) CLOTHING ARTICLE HAVING AN INTEGRATED ABSORBENT MATERIAL

(71) Applicant: Josephine Kwan, Vancouver (CA)

(72) Inventor: Josephine Kwan, Vancouver (CA)

(21) Appl. No.: 16/397,903

(22) Filed: Apr. 29, 2019

(51) Int. Cl. A61F 13/49; A41D 1/08; A41D 31/12

(52) U.S. Cl. CPC A61F 13/49006; A41D 31/125; A41D 1/08

(57) ABSTRACT

An absorbent performance garment is disclosed comprising a pant and a gusset at least partially sewn to an interior surface of the pant. The gusset is comprised of a first layer, an absorbent second layer, and a waterproof third layer. The second layer is positioned between the first layer and the waterproof third layer. The gusset is affixed to the pant such that the gusset can absorb fluids without weighing down the crotch region of the pant.

### TECHNICAL FIELD

The present disclosure generally relates to articles of apparel and more particularly relates to pants with improved protection and performance.

### BACKGROUND

It has been shown that about 10 million women in the United States are affected by heavy or long menstrual periods (menorrhagia). Further about 20 million women in the Unites States suffer from various forms of incontinence. Current solutions include adult diapers or other absorbent articles which absorb moisture while worn.

Often, performance apparel incorporates materials which aid in wicking away sweat or other bodily fluids. The materials are provided in a blend to permit adequate range-of-motion needed during exercise activities while drawing some moisture away from the skin to manage body heat and reduce odor.

In the current arts, leggings and technical apparel such as yoga pants are specifically designed to allow maximum movement for a variety of exercise routines or casual use. Blends of cotton, lycra spandex, nylon, polyester, wool, or similarly light and stretchy materials are employed to give the garment a soft and smooth finish.

Conventional “period proof” underwear may reduce the risk of leakage but is unattractive underneath a pair of leggings or pants as it does not conceal the visible bulky underwear lines.

While useful in providing a stylish alternative to traditional pants while also allowing sufficient movement to perform a variety of tasks and exercises, leggings and yoga pants on the market do not sufficiently protect against moisture that often collects in the crotch region.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein provide for an absorbent performance garment is comprising a pant and a gusset is at least partially sewn to an interior surface of the pant. The gusset is comprised of a first layer, an absorbent second layer, and a waterproof third layer. The second layer is positioned between the first layer and the waterproof third layer. The gusset is affixed to the pant such that the gusset can absorb fluids without weighing down the crotch region of the pant.

The garment can include yoga pants, leggings, or similar flexible pants, workwear, sports uniforms, and other forms of shorts and pants to allow suitable movement during exercise, or to be worn casually. The gusset is sewn into the interior of the pant such that it is concealed during use. Each layer of the pant gusset is staggered in size to prevent a visible bulge in the pant when worn. The first layer which contacts the skin of the user is larger than the absorbent second layer, the absorbent second layer is smaller than the waterproof layer.

In one aspect, the first layer is comprised of organic cotton, lyocell, and spandex. In particular, the first layer is comprised of between 50-70% lyocell, between 15-40% organic cotton, and between 1-20% spandex.

In one aspect, the absorbent second layer is comprised of bamboo charcoal and polyester. In particular, the absorbent second layer is comprised of between 40-70% bamboo charcoal and between 30-60% polyester.

In one aspect, the waterproof third layer is comprised of polyester and polyurethane. In particular, the waterproof third layer is comprised of between 60-85% polyester and 15-40% polyurethane.

In one aspect, the pants are comprised of recycled polyester and spandex. In particular, the pant may be comprised of between 70-95% recycled polyester and between 5-30% spandex.

In one aspect, an exterior surface of a rear side of the garment is comprised of a left side seam, and a right side seam. The left side seam and the right side seam extend at least partially under the buttock region.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a rear elevation view of the interior of the garment, according to some embodiments;

FIG. 2 illustrates a side elevation view of the interior of the garment, according to some embodiments;

FIG. 3 illustrates a front elevation view of the interior of the garment, according to some embodiments;

FIG. 4 illustrates an exploded view of the gussets, according to some embodiments.

FIG. 5 illustrates a rear elevation view of the exterior of the garment, according to some embodiments.

### DETAILED DESCRIPTION

The specific details of the single embodiment or variety of embodiments described herein are set forth in this application. Any specific details of the embodiments are used for demonstration purposes only, and no unnecessary limitation or inferences are to be understood therefrom.

Before describing in detail exemplary embodiments, it is noted that the embodiments reside primarily in combinations of components related to the device. Accordingly, device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Any numerical value contains certain errors necessarily resulting from the standard deviation found in their respective testing measurements. Moreover, all ranges disclosed herein are to be understood to encompass any sub-ranges subsumed therein. For example, a range of “less than 10” can include any sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume values as defined earlier plus negative values, e.g., −1, −1.2, −1.89, −2, −2.5, −3, −10, −20, −30, etc. It shall be further understood that any disclosed percentages of material composition are merely exemplary and that other percentages are envisioned.

The embodiments described herein, in general, relate to garments having an absorbent gusset. In particular, the garment includes activewear bottoms commonly known as yoga pants or leggings. The garment can comprise materials and designs that can offer improved moisture wicking, breathability, water repellency, moisture management, odor-free protection, and/or other beneficial qualities. Further, the garment can comprise natural and synthetic materials such as, for example, cotton, nylon, spandex, lycra, viscose, bamboo, polyester, rayon, polyurethane, and/or other fabrics or materials. According to implementations, the apparel can comprise or otherwise be a part of underwear, yoga wear, adult and child diapers, activewear, performance wear, military field wear, athletic attire, shorts, pants, uniforms, denim, and/or other types of apparel that can comprise textiles and/or non-woven materials configured to be odor-free, wick moisture from the skin, repel water, be flame retardant, and/or perform other functions.

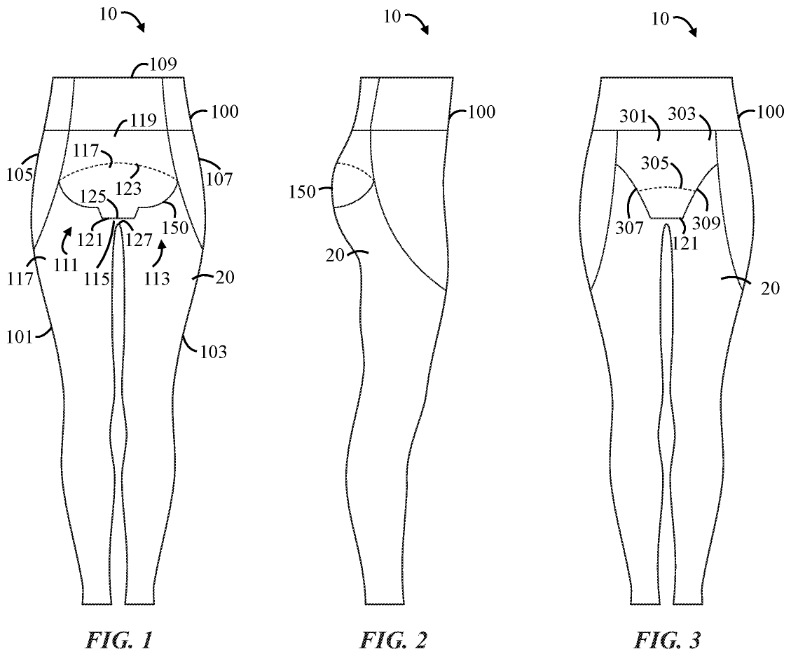

FIG. 1, FIG. 2, and FIG. 3 illustrate elevation views of the interior surface 20 of the garment 10 is shown as pants 100 having a first leg 101, a second leg 103, a first side portion 105, a second side portion 107, and a waist portion 109. As understood by those skilled in the arts, the garment 10 is dimensioned to be worn similar to a pair of leggings or yoga pants and may be provided in various sizes and configurations to facilitate use by users having various body size and configurations. Further, the garment may be form fitted, loose-fitting, flared, tapered or otherwise configured in various stylistic configurations. The first and second legs 101,103 include leg openings 111,113 wherein the users legs are inserted. A crotch region 115 is between the leg openings 111,113.

In reference to FIG. 1, the rear side 117 of the garment 10 and gusset 150 is illustrated. The rear side 117 is comprised of a top portion 119 and a crotch portion 121 having a seam 123 therebetween. The top portion 119 is bonded to the bottom of the waist portion 109. The bottom edge 125 of the crotch portion is sufficiently separated from the inner surface 127 of the crotch region 115 of the pants 100 such that the gusset 150 may absorb moisture without weighing down the crotch region 115 of the pants 100.

FIG. 3 illustrates a front elevation view of the interior surface 20 of the garment 10 and the front side of 301 of the gusset 150. The front side 301 of the gusset 150 is comprised of an upper portion 303 and the crotch portion 121 (extending continuously to the crotch portion 121 illustrated on the rear side 117 as shown in FIG. 1) separated by a midseam 305. Left and right side seams 307,309 extend from the front side 301 to the rear side 117 (see FIG. 1).

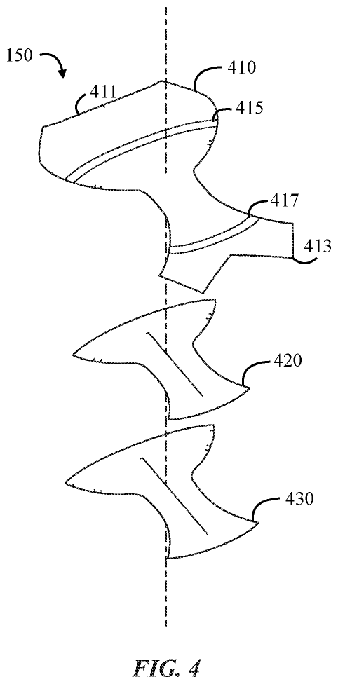

FIG. 4 illustrates an exploded view of the gusset 150 layers which are comprised of a first layer 410, an absorbent second layer 420, and a waterproof third layer 430. In some embodiments, the first layer 410 is positioned to contact the user's skin when wearing the garment. The absorbent second layer 420 is disposed between the first layer 410 and the waterproof third layer 430. The waterproof third layer 430 includes an outer surface which faces the inner surface of the pants. However, the outer surface of the waterproof third layer 430 does not contact the inner surface of the pants to prevent the pants from sagging or weighing the crotch region of the pants downward during use. In such, a substantial volume of fluid may be retained in the gusset without contacting, moistening, or weighing down the pants.

The first layer 410 may include a first end 411 and a second end 413 which extend further than the ends of the absorbent second layer 420 and the waterproof third layer 430. First and second midseams 415,417 bonds each layer of the gusset to one another and the pant.

In some embodiments, to conceal the gusset within the garment and provide a seamless appearance from the outside to not disclose the presence of the gusset within the pants, the size of each layer of the gusset is staggered. For example, the first layer 410 is larger than the absorbent second layer 420. Similarly, the absorbent second layer 420 is smaller than the waterproof third layer 430. In such, the waterproof third layer 430 conceals the first layer 410 and the absorbent second layer 420 to prevent moisture from reaching the pants.

In some embodiments, the first layer 410 is comprised of organic cotton or an organic cotton blend. In one example, the first layer 410 is comprised of lyocell, organic cotton, and spandex. In particular, the first layer 410 may utilize a blend of between 50-70% lyocell, between 15-40% organic cotton, and between 1-20% spandex. In a preferred embodiment, the first layer 410 is constructed of 66% lyocell, 28% organic cotton, and 6% spandex.

In some embodiments, the absorbent second layer 420 is comprised of bamboo charcoal and polyester. In one example, the absorbent second layer may use a blend of between 40-70% bamboo charcoal and between 30-60% polyester. In a preferred embodiment, the absorbent second layer is constructed of 52% bamboo charcoal and 48% polyester.

The absorbent second layer 420 is sufficient to wick moisture away from the user and absorb the moisture while the pants are worn. To facilitate wicking, the first layer 410 may be a wicking layer to initially transfer moisture from the user and transmit the moisture to the absorbent second layer 420. The second layer may include various hydrophilic compositions, chemicals, materials, and/or a various hydrophobic composition, chemical, or materials.

In some embodiments, the waterproof third layer 430 is comprised of polyester and polyurethane. In one example the waterproof third layer may use a blend of between 60-85% polyester and 15-40% polyurethane. In a preferred embodiment, the waterproof layer is constructed of 75% polyester and 25% polyurethane. The waterproof layer may be treated, such as with silver, to prevent the growth of bacteria and reduce odor.

One or more layer of the gusset and pants may be utilized to prevent odor along with wicking moisture away from the skin. One or more materials of the garment may be antimicrobial to prevent or substantially reduce the growth of microbes.

In some embodiments, the pants may utilize a blend of materials as described hereinabove. In one example, the pants may utilize recycled polyester, such as from recycled plastic bottles, and spandex. In a particular example, the pants utilize between 70-95% recycled polyester and between 5-30% spandex. In the preferred embodiment, the pants are constructed of 88% recycled polyester and 12% spandex. One skilled in the arts will readily understand that a variety of materials and configurations of materials well known in the arts may be used for the garment.

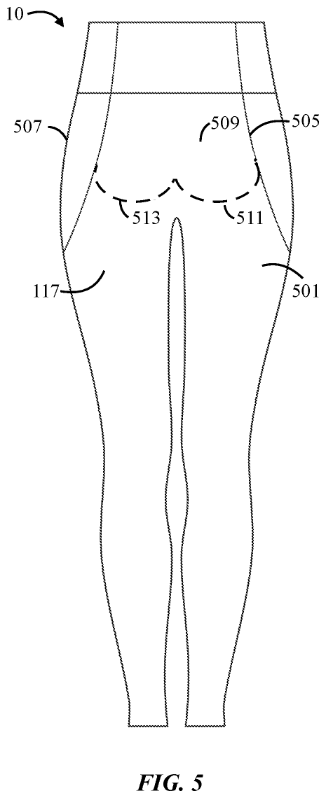

FIG. 5 illustrates a rear elevation view of the exterior 501 of the garment 10. The exterior 501 is configured to sufficiently conceal the presence of the seams which attach the gusset into the garment 10. In one example, a left bottom seam 511 and a right bottom seam 513 extend from the left region 505 and right side region 507. The left and right bottom seams 511,513 each extend under the buttock region 509.

In some embodiments, each seam includes a set of seams for each layer of the gusset. Each layer is staggered in size to conceal the presence of the gusset positioned between the user's skin and the garment. The presence of the gusset may be concealed from the wearer and observers of the wearer.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A garment having an absorbent gusset, comprising:
a. a pant;
b. a gusset portion comprised of three layers, the gusset portion at least partially sewn into a crotch region of the pant.

2. The garment of claim 1, wherein the three layers of the gusset portion are comprised of a first layer arranged to contact the user, an absorbent second layer, and a waterproof third layer, wherein the absorbent second layer is positioned between the first layer and the waterproof third layer.

3. The garment of claim 2, wherein the first layer is comprised of organic cotton, lyocell, and spandex.

4. The garment of claim 3, wherein the absorbent second layer is comprised of bamboo charcoal and polyester.

5. The garment of claim 4, wherein the waterproof third layer is comprised of polyester and polyurethane.

6. The garment of claim 1, wherein an exterior surface of a rear side of the garment is comprised of a left side seam and a right side seam, wherein the left side seam and the right side seam extend at least partially under the buttock region, wherein the left side seam and the right side seam facilitate the concealed attachment of the gusset to the garment.

7. An absorbent performance garment, comprising:
a. a pant;
b. a gusset at least partially sewn to an interior surface of the pant, the gusset comprised of a first layer, an absorbent second layer, and a waterproof third layer, wherein the second layer is positioned between the first layer and the waterproof third layer.

8. The garment of claim 7, wherein the gusset is sewn at the bottom seam of the waist portion.

9. The garment of claim 8, wherein the bottom surface of the gusset is separated from the interior surface of the pant.

10. The garment of claim 9, wherein the first layer is comprised of organic cotton, lyocell, and spandex.

11. The garment of claim 10, wherein the first layer is comprised of between 50-70% lyocell, between 15-40% organic cotton, and between 1-20% spandex.

12. The garment of claim 11, wherein the absorbent second layer is comprised of between 40-70% bamboo charcoal and between 30-60% polyester.

13. The garment of claim 12, wherein the waterproof third layer is comprised of between 60-85% polyester and 15-40% polyurethane.

14. The garment of claim 13, wherein an exterior surface of a rear side of the garment is comprised of a left side seam and a right side seam, wherein the left side seam and the right side seam extend at least partially under the buttock region, wherein the left side seam and the right side seam facilitate the concealed attachment of the gusset to the garment.

15. The garment of claim 14, wherein the absorbent second layer is suitable to absorb a bodily fluid without weighing down a crotch region of the pant.

16. The garment of claim 15, wherein the first layer is larger in size than the absorbent second layer.

17. The garment of claim 16, wherein the second layer is smaller in size than the waterproof third layer.

18. An absorbent performance garment, comprising:
a. a pant having a sewn interior gusset to absorb bodily fluids via an absorbent second layer comprised of bamboo charcoal and polyester, the absorbent second layer disposed between a first layer comprised of cotton and spandex and a waterproof third layer comprised of polyester and polyurethane, the gusset comprised of a bottom surface separated from the interior surface of the pant to deter the gusset from forcing a crotch region of the pant downward.

19. The garment of claim 18, wherein the first layer is larger in size than the absorbent second layer.

20. The garment of claim 19, wherein the second layer is smaller in size than the waterproof third layer.
